(19) United States
(12) Patent Application Publication
(10) Pub. No.: US 2004/0120917 A1
(43) Pub. Date: Jun. 24, 2004

(54) Cosmetic or dermopharmaceutical composition comprising an enzyme which is insoluble in an aqueous medium, as well as its uses

(75) Inventors: Eric Perrier, Les Cotes D'Arey (FR); Isabelle Bonnet, Lyon (FR); Delphine Rival, Ternay (FR)

Correspondence Address: MERCHANT & GOULD PC, P.O. BOX 2903, MINNEAPOLIS, MN, 55402-0903, US

(73) Assignee: COLETICA, Lyon (FR)

(21) Appl. No.: 10/359,788

(22) Filed: Feb. 7, 2003

(30) Foreign Application Priority Data
Dec. 18, 2002 (FR) 02 16134

(51) Int. Cl. A61K 38/46; A61K 7/11; A61K 38/47; A61K 38/48; A61K 7/06

(52) U.S. Cl. 424/70.14; 424/94.6; 424/94.61; 424/94.63

(57) ABSTRACT

The invention relates to a cosmetic or dermopharmaceutical composition, as well as these uses.

The invention relates mainly to a cosmetic or dermopharmaceutical composition, notably an anti-wrinkle cosmetic or dermopharmaceutical composition, comprising an enzyme which is insoluble in an aqueous medium, in admixture with at least one cosmetically or dermopharmaceutically acceptable excipient.

This composition is mainly used for limiting reactions of irritation and/or of allergy during topical use of this composition.

The invention relates to a cosmetic or dermopharmaceutical composition comprising an enzyme which is insoluble an aqueous medium, in admixture with at least one cosmetically or dermopharmaceutically acceptable excipient, as well as their uses.

The invention relates mainly to the use of a composition, as described above, for limiting reactions of irritation and/or of allergy during its topical use.

### STATE OF THE ART

During the past years, alpha-hydroxy acids (AHAs) have been mainly used as an <<anti-wrinkles>> agent in the cosmetic industry. Numerous studies have shown that due to their hydrating power, the AHAs enabled the upper layers of the epidermis to take in water, which led to a lowering of the inter-corneocytary cohesion forces (Van Scott et al. 1984, J. Am. Acad. Dermat. 11, 867-879; Zoe Draelos 2000, Cosmetic Dermatology, 10, 51-57).

Despite a very high effectiveness, the AHAs, just as other products having an intense keratolytic activity (salicylic acid, fruit acids), can trigger off violent reactions of irritation which can manifest themselves for doses which are sometimes less than those necessary to obtain a keratolytic effect (Slavin 1998, Clin. Plast. Surg. 25, 45-52).

Another approach route for developing a keratolytic active consists in using enzymes such as proteases or lipases. In fact, on the normal human skin, two enzymes having proteolytic activity are mainly responsible for the process of desquamation, the Stratum corneum chymotrypsin enzyme (SCCE) and the stratum corneum tryptic enzyme (SCTE) (Lundström et al. 1991, Acta Derm. Veneol., 41, 471-474; Ekholm et al. 2000. J. Invest. Dermatol., 114, 56-63). It therefore appears natural to use enzymes such as proteases for accelerating the phenomena of desquamation since these enzymes are physiologically present on the surface of the skin, or such as lipases or glycosidases so as to destroy the lipid or glucide organization, which enables augmenting the effects of desquamation, which, in the end, induces an intensification of the mechanisms of multiplication of the keratinocytes and induces an anti-wrinkle effect.

However, the use of enzymes is almost impossible in cosmetics : all the enzymes are unstable in an aqueous medium at the temperatures of the ageing tests which are generally used (45° C.), and all the enzymes are generally badly tolerated by the human skin (irritation and allergies are the obvious clinical signs of this intolerance). Thus, for example, the proteases are particularly unstable in aqueous media since, due to their protein structure, they undergo autolysis (they hydrolyse themselves) in the presence of water. Certain authors have proposed modifying, by directed mutagenesis, the autolysed peptide sites (Varallyay 1998, Biochem. Biophys. Res. Commun., 4, 243, 56-60; Van den Burg 1998, Biotechnol. Appl. Biochem., 27, 125-132), immobilizing the enzyme by coupling it covalently with a soluble polymer (Lee et al. 1998, Biotechnol. Prog., 14, 3, 508-516) or, even, placing the enzyme in suspension in a hydrophobic phase so as to separate aqueous phase from the emulsion (patent 97U.S. Pat. No. 866,916).

The second problem is the very high allergenicity which is caused by the application of enzymes, in particular of proteases on the surface of the skin (Pepsy et al. 1985, Clin. Allergy, 15, 101-115; Soto-Mera et al. 2000, Allergy, 55, 983-984). The phenomena of intolerance observed are probably linked to the significant penetration of soluble peptides which are present in the enzyme solutions used, whether they originate from an incomplete purification of the enzymes, or whether they originate from fragments which are produced by hydrolysis or autolysis of the enzymes used, the peptides having then a strong immunogenic property.

### AIMS OF THE INVENTION

A main aim of the invention is to solve the novel technical problem consisting of providing a cosmetic or dermopharmaceutical composition being able to be used without any problem of irritation or of allergy, when it comprises an enzyme.

Another aim of the invention is to solve the novel technical problem consisting of providing a cosmetic or dermopharmaceutical composition having an anti-wrinkle effect, comprising an enzyme.

Another aim of the invention is to solve the novel technical problem consisting of providing a cosmetic or dermopharmaceutical composition promoting an intensification of the mechanisms of multiplication of the keratinocytes, and a lightening of the skin.

Another aim of the invention is to solve the novel technical problem consisting of providing a cosmetic or dermopharmaceutical composition promoting the reconstruction of the skin barrier and comprising an enzyme.

Furthermore, an aim of the invention is to solve the novel technical problem consisting of providing a cosmetic or dermopharmaceutical composition enabling the treatment of dry or greasy skins, comprising an enzyme.

### DESCRIPTION OF THE INVENTION

The whole of these technical problems is solved for the first time simultaneously by the present invention.

Thus, within the context of the invention, it was discovered in a particularly unexpected manner that enzymes in a form which is insoluble in an aqueous medium can be used in cosmetic or dermopharmaceutical compositions, notably without problem of irritation or of allergy.

Thus, of a first aspect, the invention relates to a cosmetic or dermopharmaceutical composition comprising at least one enzyme which is insoluble in an aqueous medium, in admixture with at least one cosmetically or dermopharmaceutically acceptable excipient.

By “excipient” the inventors mean the whole of the components other than the active principle, which is the enzyme in insoluble form.

Advantageously, this enzyme is selected from the group consisting lipases, oxydoreductases, carbohydrases and proteases.

Advantageously, this enzyme is selected from the group consisting of a protease such as subtilisin or trypsin or chymotrypsin or thermolysine, a lipase, a phospholipase, an amylase such as alpha-amylase or beta-amylase or glucoamylase, beta D-Glucosidase, cerebrosidase, a superoxide dismutase, a peroxidase, and a lipoxygenase.

From the whole of the embodiments that this invention covers, the inventors recommend three particular embodiments of this invention:

a first advantageous embodiment consists in that the enzyme is crystallized so as to form insoluble protein crystals, and then these crystals are cross-linked chemically, e.g. by glutaraldehyde, so as to insolubilize these particles and to render them insoluble in aqueous media;

a second advantageous embodiment consists in that the enzyme is grafted onto a polymer which is selected in such a way that it be insoluble in an aqueous medium;

a third advantageous embodiment consists in that the enzyme is grafted onto particles, preferably micrometric or nanometric particles, preferably the particles being spheres, capsules or sponges, which are all insoluble in an aqueous medium.

Advantageously, these polymers or these particles, which are insoluble in an aqueous medium, have on their surface at least one modifiable chemical function, which is capable of being used for forming a covalent bond with the enzyme, e.g. these polymers or these particles comprise at least one of the following: one cellulose, one polystyrene, one alkylcyanoacrylate, one silica, one nylon, one polyamide (synthetic or originating from a natural polyamide), one polyester (synthetic or originating from a natural polyester), or one of their mixtures.

Within the context of this invention, the spheres can be spheres as described in the patents U.S. Pat. No. 5,395,620; FR 2,683,159 (U.S. Pat. No. 6,303,150); WO 94/04261 (U.S. Pat. No. 5,691,060); U.S. Pat. No. 5,912,016; FR 2,780,901 (U.S. Pat. No. 6,197,757); FR 2,703,927 (U.S. Pat. No. 5,635,609).

Of the first embodiment, the crystallized and cross-linked enzyme is in a form of crystals. These crystals have a size of between 0.2 and 50 microns, preferably of between 1 and 5 microns. These crystals notably have needle or ovoid forms and the size given corresponds to their largest dimension.

These crystals are formed notably by the technique of insolubilization of crystals of enzymes by cross-linking as described in other respects (<<Cross-link enzyme crystal>>, TIBTECH, 1996, 14, 7(150), 219-259).

These crystals are preferably diluted in a gel, notably a gel which is acceptable for the skin and/or the scalp, and/or the hair, so as to prepare a cosmetic or dermopharmaceutical composition.

Advantageously, the excipient contains at least one compound selected from the group consisting of butylene glycol, water, steareth-2, steareth-21, glycol-15 stearyl ether, cetearyl alcohol, phenoxyethanol, methylparaben, ethylparaben, propylparaben, butylparaben, butylene glycol, natural tocopherols, glycerol, sodium dihydroxycetyl, isopropyl hydroxycetyl ether, glycol stearate, triisononaoine, octyl cocoate, polyacrylamide, isoparaffin, laureth-7, a carbomer, propylene glycol, glycerol, bisabolol, dimethicone, sodium hydroxide, a perfume, PEG 30-dipolyhydroxystearate, capric/caprylic triglycerides, cetearyl octanoate, dibutyl adipate, grape seed oil, jojoba oil, magnesium sulfate, EDTA, a cyclomethicone, xanthan gum, citric acid, sodium lauryl sulfate, mineral waxes and oils, isostearyl isostearate, propylene glycol dipelargonate, propylene glycol isostearate, PEG 8 Beeswax, hydrogenated palm tree heart oil glycerides, hydrogenated palm oil glycerides, lanolin oil, sesame oil, cetyl lactate, lanolin alcohol, castor oil, titanium dioxide, colorants, and pigments.

Advantageously, the composition cited above is formulated in a form selected from the group consisting of a solution, which is aqueous or oily, an aqueous cream or gel or an oily gel, notably in a pot or in a tube, notably a shower gel, a shampoo; a milk; an emulsion, a microemulsion or a nanoemulsion, notably an oil-in-water or water-in-oil or multiple or silicone-containing microemulsion or nanoemulsion; a lotion, notably in a glass bottle, a plastic bottle or in a measure bottle or in an aerosol; an ampoule; a liquid soap; a dermatological bar; an ointment; a foam; an anhydrous product, preferably a liquid, pasty or solid anhydrous product, e.g. in the form of a stick, notably in the form of a lipstick.

Advantageously, the enzyme is grafted by a covalent bond onto a particle or onto a polymer which is insoluble in an aqueous phase.

Advantageously, the enzyme is grafted onto a sphere, the sphere is prepared to react with the enzyme to be grafted by activation with a bifunctional agent, such as a carbodiimide.

In general, the polymers or the particles used during the grafting of the enzyme onto these particles can be activated by an activating agent. This activation consists mainly of the activation of the chemical functions which are present on the external surface of the polymer or of the particle.

Advantageously, the crystallized and cross-linked enzyme is cross-linked by glutaraldehyde.

In general, and unexpectedly, the inventors, during the realization of this invention, have also shown that during the steps of insolubilization of the enzyme, a purification step was obtained and that allergenic enzymes used in a soluble form became totally hypoallergenic once used in their insoluble forms.

Furthermore, the use of the enzyme in insoluble form enables stabilizing the enzymatic activities of the enzymes thus modified, and this enables envisaging their use in cosmetic and dermopharmaceutical applications.

The enzyme which is rendered insoluble in an aqueous medium enables a systematic improvement of the skin tolerance and the possibility of using these enzymes in cosmetic or dermopharmaceutical formulations, notably hypoallergenic cosmetic or dermopharmaceutical formulations, whereas usually they cannot be used.

Unexpectedly, when the cosmetic or dermopharmaceutical composition described above comprises at least one active principle (other than the enzyme in insoluble form), the enzyme enables the improvement of the trans-cutaneous penetration of at least this active principle.

Thus, the invention relates, of a second aspect, to the use of an enzyme which is insoluble in an aqueous medium, preferably in admixture with a cosmetically or dermopharmaceutically acceptable excipient so as to form a cosmetic or dermopharmaceutical composition as defined above, for carrying out a cosmetic or dermopharmaceutical care.

Advantageously, this enzyme, which is preferably in admixture in order to form a cosmetic or dermopharmaceutical composition, can be used for limiting reactions of irritation and/or of allergy during its topical use.

Advantageously, the use of this enzyme, or of a cosmetic or dermopharmaceutical composition containing it, enables making an intensification of the mechanisms of multiplication of the keratinocytes, notably of the keratin of the skin and/or of the hair, notably enabling a lightening of the skin, preferably this enzyme is a protease.

Advantageously, this enzyme, or a cosmetic or dermopharmaceutical composition containing it, can be used to make an anti-wrinkle effect.

Advantageously, this enzyme, or a cosmetic or dermopharmaceutical composition containing it, can be used for the reconstruction of the skin barrier so as to obtain a barrier effect, this enzyme is preferably a lipase or an amylase.

Advantageously, this enzyme, or a cosmetic or dermopharmaceutical composition containing it, enables the elimination of the excess of sebum and the disappearance of the shiny effect of the skin, by topical application on a greasy skin, this enzyme is preferably a lipase.

Advantageously, this enzyme, or a cosmetic or dermopharmaceutical composition containing it, enables the disappearance of squamae, and the return to a normal state by topical application on a dry skin, this enzyme is preferably a protease or an amylase.

Advantageously, a cosmetic or dermopharmaceutical composition containing this enzyme and at least one active principle (other than the enzyme in insoluble form), enable increasing the trans-cutaneous penetration of at least this active principle contained in this composition.

Of a third aspect, the invention relates to a method of cosmetic care comprising topically applying an enzyme or a cosmetic composition as defined above.

Of a fourth aspect, the invention relates to a dermopharmaceutical care method comprising topically applying an enzyme or a dermopharmaceutical composition as defined above.

In particular, for all the aspects of the invention, this topical application relates to an external application notably on the skin, and/or the scalp, and/or the hair.

Other aims, characteristics and advantages of the invention will appear clearly to the person skilled in the art upon reading the explanatory description which makes reference to the Examples which are given simply as an illustration and which in no way limit the scope of the invention.

The Examples make up an integral part of the present invention, and any characteristic which appears novel with respect to any prior state of the art from the description taken in its entirety, including the Examples, makes up an integral part of the invention in its function and in its generality.

Thus, every example has a general scope.

Furthermore, in the Examples, all percentages are given by weight, unless indicated otherwise, ambient temperature is expressed in degrees Celsius unless indicated otherwise, and the pressure is atmospheric pressure, unless indicated otherwise.

### EXAMPLES

### Example 1

Protease in Insoluble Crystallized Form.

Subtilisin is first of all crystallized which enables eliminating the impurities, and then the crystalline form is stabilized by virtue of a cross-linking of the proteins by glutaraldehyde. The technique used is that described in various publications such as TIBTECH, 1996, 14, 7(150), 219-259.

The protein crystals thus obtained are perfectly insoluble in an aqueous medium. By this process, the interactions of the enzyme molecules between themselves are reduced and the number of cleavage sites which are recognized by the active site of the enzymes is reduced. The autolysis of the proteases is thus reduced, which thus enables obtaining a very good enzymatic stability in an aqueous medium.

The crystals thus obtained have a size of between 0.2 and 50 μm, and are therefore incapable of penetrating into the deep layers of the skin tissue, which limits or eliminates reactions of intolerance which are classically observed when free enzymes are used.

A gel is formed by diluting the crystals to a concentration of 10% in a 0.5% xanthan gel pre-buffered at pH=5.8 (100 mM sodium acetate buffer, CaCl₂ 20 mM). The whole is left under mechanical agitation for 10 minutes and then 2% of preservative are added so as to bacteriologically stabilize the product.

The effectiveness of the invention was first of all estimated through a test with DHA and in comparison with a commonly used α-hydroxy acid, glycolic acid. Briefly, the principle of the test is the following: 4 areas of the forearm of 20 volunteers are treated with the aid of a cosmetic preparation containing 5% of DHA, 2 times per day for 3 days. An intense coloration resulting from the reaction of the DHA with the skin proteins is induced; under the daily application of various keratolytic formulations, this coloration which disappears can be followed and compared, in using a chromametric method (Minolta Chromameter).

The use of a formulation containing 2% of the gel described above enables lowering the melanic index by 16% with respect to a control area. This lowering is 183% greater than that observed after treatment with a placebo cream and 128% greater than that observed after treatment with a cream containing 3% of glycolic acid. A formulation containing the invention is therefore twice as effective as a formulation containing 3% of glycolic acid, a keratolytic active well known for its exfoliating properties.

The anti-wrinkle effect which must thus logically arise from the keratolytic effect described above was evaluated in a second series of experiments. After application on 20 volunteers for 28 days of a formulation containing 2% of gel prepared of Example 1, the appearance and the evolution of wrinkles, which are studied by silicone molds in the <<goosefoot>>, were greatly reduced in comparison to the control area having received a placebo formulation (-30% after 15 days of treatment).

Evaluation of the Skin Sensitization Potential in the Guinea Pig:

The gel described above is subjected to the maximization test described by Magnusson and Kligmann, a protocol in agreement with the directive line No. 406 of the OECD.

Used as such (100%), the gel is classed as being non-sensitizing by contact with the skin (hypoallergenic, class I) whereas the same enzyme used in a form as such (i.e. in a non-insolubilized form), of this test, is classed as being <<very sensitizing >>.

### Example 2

Determination of the Protease Activity of the Insoluble Crystals after Incorporation in a Cosmetic Gel

2.1. So as to reproduce as accurately as possible the situation encountered in vivo, the inventor uses a protease activity determination which makes use of a substrate of high molecular weight: casein.

In practice, a solution of casein of concentration 0.66% (w/v) diluted in a 50 mM potassium phosphate buffer, pH=7.5, is placed in the presence of the insoluble cross-linked crystals of Example 1 of the invention, or of a commercially available protease, which are placed in suspension in a gel (as described in Example 1) (dilution in a 10 mM sodium acetate buffer/5 mM calcium acetate, pH=7.5).

After incubation for 10 minutes at 37° C., the free amino acids in the reaction medium during the enzymatic reaction are recovered by filtration and are then determined with the aid of Folin's reagent. This reagent absorbs at 660 nm when it is reduced notably by tyrosine, tryptophan, cysteine and histidine. The enzymatic activity is then directly proportional to the absorbance at 660 nm of the reaction medium (Folin et al. 1929, J. Biol. Chem, 73, 627; Anson et al. 1938, J. Gen. Physiol., 22, 79-89). the results are indicated in Table 1.

TABLE 1

| Composition of the | |
|---|---|
| sample | Activity in U/g |
| Cross-linked subtilisin crystals | 1.33 U/g |
| of Example 1 | |
| Free subtilisin (Bacillus | 0.62 U/g |
| licheniformis) | |
| [C] = 0.0061% | |

2.2 A gel prepared of Example 1, containing the insoluble cross-linked crystallized subtilisin (hereafter called “subtilisin crystals”), and a gel containing commercially available subtilisin (extract from Bacillus licheniformis) are placed at 4° C., 20° C. and 45° C. The determination of the protease activity is carried out of the technique described above. The results, which are given in % of found activity versus the initial activity at T=0, are indicated in the Tables below.

| | Subtilisin crystals | Free subtilisin |
|---|---|---|
| T = 1 month | | |
| 4° C. | 104.5 | 98.5 |
| 20° C. | 103.6 | 93 |
| 45° C. | 98.4 | 51.5 |
| T = 6 months | | |
| 4° C. | 135.3 | 79.8 |
| 20° C. | 126.8 | 53.2 |
| 45° C. | 90.5 | 4.9 |
| T = 12 months | | |
| 4° C. | 130 | 72 |
| 20° C. | 139 | 34.6 |
| 45° C. | 47.5 | 0 |

The invention therefore possesses a very high enzymatic stability, whatever the storage temperature be.

### Example 3

Other Cross-Linked and Crystallized Enzymes

Lipases and glucosidases can also be purified and then crystallized, and then be cross-linked chemically in order to obtain insoluble particles.

Thus, it has been possible for lipases or amylases, which are made by fermentations, to be insolubilized as crystals and then used for cosmetic applications.

The cosmetic effects obtained were evaluated on series of 15 volunteers. The grades were made by a dermatological expert led to a grading of the anti-wrinkle results, greasy skins, and skins normalized after an aggression with 10% of sodium lauryl sulfate. The grading is the following: from non-effective (−) to very effective (+++); nuh: non-usable on humans due to allergy problems:

| | | Anti- | Barrier | Greasy |
|---|---|---|---|---|
| | Tolerance | wrinkle | effect | skins effect |
| Free lipase | Severe allergies | Nuh | nuh | nuh |
| Insolubilized | Perfectly | +++ | +++ | +++ |
| lipase | tolerated | | | |
| Free | Severe allergies | Nuh | nuh | nuh |
| phospholipase | | | | |
| Insolubilized | Perfectly | ++ | ++ | ++ |
| phospholipase | tolerated | | | |
| | | Anti- | Barrier | Dry |
| | Tolerance | wrinkle | effect | skins effect |
| Free alpha | Severe allergies | nuh | nuh | nuh |
| amylase | | | | |
| Insolubilized | Perfectly | + | ++ | ++ |
| alpha amylase | tolerated | | | |
| Free beta | Severe allergies | nuh | nuh | nuh |
| amylase | | | | |
| Insolubilized | Perfectly | ++ | ++ | + |
| beta amylase | tolerated | | | |
| Free | Severe allergies | nuh | nuh | nuh |
| glucoamylase | | | | |
| Insolubilized | Perfectly | +++ | ++ | ++ |
| glucoamylase | tolerated | | | |
| Free | Severe allergies | nuh | nuh | nuh |
| cerebrosidase | | | | |
| Insolubilized | Perfectly | ++ | +++ | +++ |
| cerebrosidase | tolerated | | | |

### Example 4

Grafting of Enzymes onto Particles which are Insoluble in the Aqueous Phase

Cerebrosidase can be grafted onto particles in the following way: 10 g of spheres prepared as described in the COLETICA patents U.S. Pat. No. 5,395,620; FR 2,683,159 (U.S. Pat. No. 6,303,150); WO 94/04261 (U.S. Pat. No. 5,691,060); U.S. Pat. No. 5,912,016; FR 2,780,901 (U.S. Pat. No. 6,197,757), FR 2,703,927 (U.S. Pat. No. 5,635,609) are placed to react with carboxydiimide (from 1 to 30 g for log of spheres) in an aqueous solution kept at a pH of between 4 and 8 so as to activate the formation of <<reactive>> carboxylic groups. The enzymes that are desired to graft are then placed in contact with the reaction product and after grafting for 1 to 24 hours, at a temperature of between 4 and 60° C, the reaction product is rinsed by simple filtration or decantation and is then separated from its reaction medium, before being placed in suspension in a gel in order to facilitate its marketing.

In the same way, it has been possible for lipases, proteases and amylases, which are prepared by fermentations, to be insolubilized in the form of insoluble particles, and then used for cosmetic applications.

The cosmetic effects obtained were evaluated on series of 15 volunteers. The grades were made by a dermatological expert led to a grading of the anti-wrinkle results, greasy skins, and skins normalized after an aggression with 10% sodium lauryl sulfate. The grading is the following: from non-effective(−) to very effective (+++); nuh: non-usable on humans due to allergy problems

| | | Anti- | Barrier | Greasy skins |
|---|---|---|---|---|
| | Tolerance | wrinkle | effect | effect |
| Free lipase | Severe | nuh | Nuh | nuh |
| | allergies | | | |
| Insolubilized | Perfectly | +++ | +++ | +++ |
| lipase | tolerated | | | |
| Free | Severe | nuh | Nuh | nuh |
| phospholipase | allergies | | | |
| Insolubilized | Perfectly | ++ | ++ | ++ |
| phospholipase | tolerated | | | |
| | | Anti- | Barrier | Dry |
| | Tolerance | wrinkle | effect | skins effect |
| Free alpha | Severe | nuh | Nuh | nuh |
| amylase | allergies | | | |
| Insolubilized | Perfectly | + | + | + |
| alpha amylase | tolerated | | | |
| Free beta | Severe | nuh | Nuh | nuh |
| amylase | allergies | | | |
| Insolubilized | Perfectly | ++ | ++ | + |
| beta amylase | tolerated | | | |
| Free | Severe | nuh | Nuh | nuh |
| glucoamylase | allergies | | | |
| Insolubilized | Perfectly | +++ | ++ | +++ |
| glucoamylase | tolerated | | | |
| Free | Allergys | nuh | Nuh | nuh |
| cerebrosidase | Severes | | | |
| Insolubilized | Perfectly | ++ | ++ | +++ |
| cerebrosidase | tolerated | | | |

### Example 5

Grafting of Beta-D Glucosidase onto Particles which are Insoluble in an Aqueous Phase

### Example 5a

Beta-D glucosidase extracted from sweet almond (Sigma) is grafted onto insoluble particles of a protocol comprising 3 phases:

A phase of activation of the carboxylic functions of the proteins used for the preparation of insoluble particles which are prepared of the COLETICA patent U.S. Pat. No. 5,395,620. This step is carried out by adding 0.4 g of 1-ethyl-3-(3-dimethylaminopropyl)carbodiimide (Sigma) dissolved in a 10 mM hepes buffer, pH 7.5, to 100 grams of microspheres as a plug. This solution is agitated for 1 hour at ambient temperature, and then the spheres are recovered after centrifugation at 2,300 rpm for 3 minutes. The plug is rinsed thrice with 10 mM hepes buffer, pH 7.5.

A phase of coupling with the enzyme, wherein the beta-D glucosidase is added in solution at a content of 0.04% in 0.1M carbonate buffer, pH 8.5.

The solution is agitated for 1 hour at ambient temperature.

A phase of rinsing, in order to eliminate the free enzyme: the solution is centrifuged at 2,300 rpm for 3 minutes and then the supernatant is eliminated. The spheres which are grafted with the enzyme are rinsed thrice with 0.1M acetate buffer, pH 6.2.

The rinsed grafted spheres are placed in solution at a content of 20% in a 0.1M acetate buffer medium, pH 6.2.

Demonstration of the Stabilization of the Enzyme by Grafting:

The product originating from Example 5a is determined in terms of beta D-Glucosidase activity and is then placed at 20° C. and 45° C. for a study of the stability of the enzymatic activity with time at these 2 temperatures. A sample constituted by the free enzyme, beta D-Glucosidase, from sweet almond, placed in solution in 0.1M acetate buffer, pH 6.2 at the same concentration, is made so as to test the stability at 20° C. and 45° C.

Principle of the Determination:

The measurement of the beta D-Glucosidase activity is done by a fluorimetric technique using a probe, 4-methylumbelliferyl β-D-Glucopyranoside (Molecular Probes), which, hydrolyzed by the enzyme, enables releasing the oligosaccharide and 4-methylumbelliferone, a compound which is fluorescent at excitation and emission wavelengths of 360/460 nm, respectively.

The results are expressed in % of activity with respect to the activity measured at t=0.

Grafted Enzyme (Example 5a)

| Time | Activity at 20° C. | Activities at 45° C. |
|---|---|---|
| (days) | (%) | (%) |
| 0 | 100 | 100 |
| 5 | 100 | 88.5 |
| 12 | 100 | 24 |
| 19 | 100 | 11.5 |

Free Enzyme in Acetate Buffer:

| Time | Activity at 20° C. | Activities at 45° C. |
|---|---|---|
| (days) | (%) | (%) |
| 0 | 100 | 100 |
| 5 | 72 | 28 |
| 12 | 59 | 6 |
| 19 | 52.5 | 4 |

The grafting of the enzyme onto the spheres has enabled stabilizing the enzymatic activity at 20° C., since 100% activity is obtained after 1 month at 20° C., and has enabled slowing down the loss of activity at 45° C. with respect to that observed for the free enzyme.

Stability of the Samples After Incorporation in a Formulation

The cream used is made up of:

| | | |
|---|---|---|
| Aqueous phase: | Butylene Glycol | 4% |
| | Water | qsp 100 |
| Oily phase: | Steareth-2 | 3% |
| | Steareth-21 | 2% |
| | Glycol-15 Stearyl Ether | 9% |
| | Cetearyl Alcohol | 2.5% |
| Then addition | Phenoxyethanol, Methylparaben, Ethylparaben | 0.5% |
| of: | Propylparaben, Butylparaben | |
| | Butylene Glycol | 0.5% |
| | D Mixed tocopherols | 0.2% |
| | Product of the invention | 5% |

The measurement of the beta D-Glucosidase activity of the samples incorporated in a cream is made directly on the aqueous phase, which is obtained after separation of the aqueous and oily phases of the following protocol: the cream is diluted 5 times in a 0.1M citric acid buffer/di-Na hydrogenophosphate 0.2M, pH 5. 10% of NaCl are then added and the mixture is subjected to a very strong agitation for 5 minutes and is then centrifuged at 5,000 rpm for 25 minutes. The measurement of the activity is carried out directly on the aqueous phase obtained.

Stability of the Cream Containing the Sample Obtained of Example 5a or the Free Enzyme:

| | Example 5a | Free enzyme |
|---|---|---|
| Temps | Activity at 20° C. | Activity at 20° C. |
| (days) | (%) | (%) |
| 0 | 100 | 100 |
| 8 | 97 | 10 |
| 15 | 43.5 | 2 |
| 22 | 99.5 | 0 |
| 31 | 88 | 0 |

The enzymatic activity measured in the formulations at 20° C. remains stable since around 90% of activity is conserved after 1 month at 20° C. for the enzyme grafted onto the spheres.

### Example 5b

Beta D-glucosidase or any other commercially available enzyme is grafted onto insoluble particles of the protocol of Example 5a above, but in using insoluble particles of the following types: cellulose beads, polystyrene beads, alkylcyanoacrylate beads, nylon beads, silica beads, polyamides beads, polyester beads, or any bead having on its surface a modifiable chemical function which is capable of being used for forming a covalent bond with a chemical function of an enzyme, in preferably using an agent which is either capable of activating these chemical functions, or a bifunctional agent enabling the reaction between the chemical functions described above.

### Example 6

Use of the Products of the Invention in Cosmetic or Pharmaceutical Formulations of the Oil in Water Emulsion Type

Formulation 6a:

| A | water | qsp 100 |
|---|---|---|
| | Butylene Glycol | 2 |
| | Glycerol | 3 |
| | Sodium Dihydroxycetyl | 2 |
| | Phosphate, | |
| | Isopropyl Hydroxycetyl Ether | |
| B | Glycol Stearate SE | 14 |
| | Triisononaoin | 5 |
| | Octyl Cocoate | 6 |
| C | Butylene Glycol, | 2 |
| | Methylparaben, | |
| | Ethylparaben, Propylparaben, | |
| | pH adjusted to 5.5 | |
| D | Products of the invention | 0.0001-10% |

Formulation 6b:

| | | |
|---|---|---|
| A | Water | qsp 100 |
| | Butylene Glycol | 2 |
| | Glycerol | 3 |
| | Polyacrylamide, Isoparaffin, | 2.8 |
| | Laureth-7 | |
| B | Butylene Glycol, | 2 |
| | Methylparaben, | |
| | Ethylparaben, Propylparaben; | |
| | Phenoxyethanol, | 2 |
| | Methylparaben, | |
| | Propylparaben, Butylparaben, | |
| | Ethylparaben | |
| | Butylene Glycol | 0.5 |
| D | Products of the invention | 0.0001-10% |

Formulation 6c:

| | | |
|---|---|---|
| A | Carbomer | 0.50 |
| | Propylene Glycol | 3 |
| | Glycerol | 5 |
| | Water | qsp 100 |
| B | Octyl Cocoate | 5 |
| | Bisabolol | 0.30 |
| | Dimethicone | 0.30 |
| C | Sodium Hydroxide | 1.60 |
| D | Phenoxyethanol, | 0.50 |
| | Methylparaben, | |
| | Propylparaben, Butylparaben, | |
| | Ethylparaben | |
| E | Perfume | 0.30 |
| F | Products of the invention | 0.0001-10% |

### Example 7of the Invention

Use of the Products of the Invention in a Formulation of Water in Oil Type

| | | |
|---|---|---|
| A | PEG 30 - | 3 |
| | dipolyhydroxystearate | |
| | Capric Triglycerides | 3 |
| | Cetearyl Octanoate | 4 |
| | Dibutyl Adipate | 3 |
| | Grape Seed Oil | 1.5 |
| | Jojoba Oil | 1.5 |
| | Phenoxyethanol, | 0.5 |
| | Methylparaben, | |
| | Propylparaben, Butylparaben, | |
| | Ethylparaben | |
| B | Glycerin | 3 |
| | Butylene Glycol | 3 |
| | Magnesium Sulfate | 0.5 |
| | EDTA | 0.05 |
| | Water | qsp 100 |
| C | Cyclomethicone | 1 |
| | Dimethicone | 1 |
| D | Perfume | 0.3 |
| E | Products of the invention | 0.0001-10% |

### Example 8 of the Invention

Use of the Products of the Invention in a Formulation of Shamooo or Shower Gel Type

| | | |
|---|---|---|
| A | Xantham Gum | 0.8 |
| | water | qsp 100 |
| B | Butylene Glycol, | 0.5 |
| | Methylparaben, | |
| | Ethylparaben, Propylparaben | |
| | Phenoxyethanol, | 0.5 |
| | Methylparaben, | |
| | Propylparaben, Butylparaben, | |
| | Ethylparaben | |
| C | Citric acid | 0.8 |
| D | Sodium Laureth Sulfate | 40.0 |
| E | Product of the invention | 0.0001-10% |

### Example 9 of the Invention

Use of the Products of the Invention in a Formulation of Lipstick Type and other Anhydrous Products

| | | |
|---|---|---|
| A | Mineral Wax | 17.0 |
| | Isostearyl Isostearate | 31.5 |
| | Propylene Glycol Dipelargonate | 2.6 |
| | Propylene Glycol Isostearate | 1.7 |
| | PEG 8 Beeswax | 3.0 |
| | Hydrogenated Palm Kernel Oil | 3.4 |
| | Glycerides, | |
| | Hydrogenated Palm Glycerides | |
| | Lanolin Oil | 3.4 |
| | Sesame Oil | 1.7 |
| | Cetyl Lactate | 1.7 |
| | Mineral Oil, Lanolin Alcohol | 3.0 |
| B | Castor Oil | Qsp 100 |
| | Titanium Dioxide | 3.9 |
| | CI 15850:1 | 0.616 |
| | CI 45410:1 | 0.256 |
| | CI 19140:1 | 0.048 |
| | CI 77491 | 2.048 |
| C | Products of the invention | 0.0001-5% |

### Example 10 of the Invention

Use of the Products of the Invention in a Formulation of Aqueous Gels (Eye Surrounds, Slimmers, etc . . . )

| A | water | Qsp 100 |
|---|---|---|
| | Carbomer | 0.5 |
| | Butylene Glycol | 15 |
| | Phenoxyethanol, Methylparaben, | 0.5 |
| | Propylparaben, Butylparaben, | |
| | Ethylparaben | |
| B | Products of the invention | 0.0001-10% |

### Example 11

Evaluation of the Cosmetic Acceptance of a Preparation Containing an Insoluble Enzyme of the Invention

Toxicology tests were carried out on the compound obtained of Example 1 by an ocular evaluation in the rabbit, by the study of the absence of abnormal toxicity by single oral administration in the rat and by the study of the sensitizing power in the guinea pig. A study of hypoallergenicity on human volunteers was then made with a preparation comprising the compound described in Example 1 diluted to 10% in a 0.45% carbopol gel, pH=5.8.

Evaluation of the Primary Irritation of the Skin in the Rabbit:

The preparation described above is applied without dilution at the dose of 0.5 ml on the skin of 3 rabbits of the method recommended by the OECD in relation to the study of <<the acute irritant/corrosive effect on the skin>>.

The products are classed of the criteria defined in the Decision of 20/04/99 taken in application of the basic directive 67/548/EEC and its successive amendments.

The preparation thus tested is not classed amongst the products which are irritant for the skin.

Evaluation of the Ocular Irritation in the Rabbit:

The preparation described above was instilled pure and in one batch at the rate of 0.1 ml in the eye of three rabbits of the method recommended by the basic directive 67/548/EEC and its successive amendments.

The results of this test enable concluding that the preparation is not classed amongst the products which are irritant for the eyes.

Test on the Absence of Abnormal Toxicity by Single Oral Administration in the Rat:

The preparations described were administered in one batch orally at the dose of 5 g/Kg of body weight, to 5 male rats and 5 female rats of a protocol inspired from the directive of the OECD No. 401 of 24^{th} February 1987 and adapted to cosmetic products.

The LD0 and LD50 are found to be greater than 5,000 mg/Kg. The preparation tested is therefore not classed amongst the preparations which are dangerous by ingestion.

Evaluation of the Skin Sensitization Potential in the Guinea Pig:

The preparation described is subjected to the maximization test described by Magnusson and Kligmann, a protocol which is in agreement with the directive line No. 406 of the OECD.

The preparation is classed as non-sensitizing by contact with the skin.

Hypoallergenicity Test on Human Volunteers

The hypoallergenicity of the product was tested on the product described in Example 1 and was diluted to 10% in a gel. The test was carried out on a panel of 100 healthy volunteers.

The product is applied under an occlusive patch for 24 hours, and is then re-applied under patch for 2 days for a total of 9 applications (induction phase). After a period of 2 weeks, other patches containing the product are applied onto the skin of the volunteers and are left in contact for 24 hours. The clinical signs of irritation and skin sensitization are evaluated 24, 48 and 72 hours after the removal of the patch (challenge phase).

Under these experimental conditions, the product was shown to be devoid of allergenic potential.

### Example 12

Increase in the Penetration of Ascorbic Acid Contained in a Cosmetic Formulation, in the Presence of the Product of the Invention

The tests were carried out on the formulation 6a, in which the product of the invention (D) was replaced by

Formula A: 2% of the product of the invention as described in Example 1, and 2% of ascorbic acid

Formula B : 2% of ascorbic acid only.

The two formulations were deposited (50 μg/cm²) on a human biopsy mounted on diffusion cells. The amount of ascorbic acid capable of traversing the biopsy is quantified in the receiving compartment of the diffusion cell, and is quantified by HPLC.

After 24 hours of diffusion, the amount of ascorbic acid which is capable of being found in the receiving compartment is 4 times greater for the formulation A than for the formulation B. The product of the invention thus enables a better penetration of the ascorbic acid present in the formulation; it is therefore an agent which promotes the penetration of active principles.

### Example 13

Increase of the Penetration of Caffeine Contained in a Cosmetic Formulation, in the Presence of the Product of the Invention.

The tests were carried out on the formulation 6a, in which the product of the invention (D) was replaced by:

Formula A: 2% of the product of the invention as described in Example 1, and 3% of caffeine,

Formula B: 3% of caffeine only.

The two formulations were deposited (50 μg/cm²) on a human biopsy mounted on diffusion cells. The amount of caffeine capable of traversing the biopsy is quantified in the receiving compartment of the diffusion cell, and is quantified by HPLC.

After 24 hours of diffusion, the amount of caffeine which is capable of being found in the receiving compartment is 47% greater for the formulation A than for the formulation B. The product of the invention thus enables a better penetration of the caffeine present in the formulation; it is therefore an agent which promotes the penetration of active principles.

What is claimed is:

1. A composition selected from the group consisting of: a cosmetic composition, comprising at least one enzyme which is insoluble in an aqueous medium, in admixture with at least one cosmetically acceptable excipient; and a dermopharmaceutical composition, comprising at least one enzyme which is insoluble in an aqueous medium, in admixture with at least one dermopharmaceutically acceptable excipient.

2. The composition of claim 1, wherein the enzyme is selected from the group consisting of: a lipase, an oxydoreductase, a carbohydrase, and a protease.

3. The composition of claim 1, wherein the enzyme is selected from the group consisting of: a protease, a lipase, a phospholipase, an amylase, beta D-Glucosidase, cerebrosidase, a superoxide dismutase, a peroxidase, a lipoxygenase, subtilisin, trypsin, chymotrypsin, thermolysine, alpha-amylase, beta-amylase, and glucoamylase.

4. The composition of claim 1, wherein the enzyme is selected from the group consisting of: a crystallized and cross-linked enzyme, an enzyme grafted onto a water insoluble polymer, and an enzyme grafted onto water insoluble particles.

5. The composition of claim 4, wherein said particles are selected from the group consisting of: micrometic particles, and nanometric particles.

6. The composition of claim 4, wherein said particles are selected from the group consisting of: spheres, capsules, and sponges.

7. The composition of claim 4, wherein said insoluble polymers or particles have on their surface at least one modifiable chemical function which is capable of being used for forming a covalent bond with the enzyme.

8. The composition of claim 7, wherein said polymers or particles comprise at least one compound selected from the group consisting of: a cellulose, a polystyrene, an alkylcyanoacrylate, a silica, a nylon, a synthetic polyamide, a polyamide originating from a natural polyamide, a synthetic polyester, a polyester originating from a natural polyester, and one of their mixtures.

9. The composition of claim 4, wherein the crystallized and cross-linked enzyme forms crystals having a size ranging from 0.2 to 50 μm.

10. The composition of claim 9, wherein said crystallized and cross-linked enzyme forms crystals having a size ranging from 1 to 5 μm.

11. The composition of claim 1, wherein said excipient contains at least one compound selected from the group consisting of: butylene glycol, water, steareth-2, steareth-21, glycol-15 stearyl ether, cetearyl alcohol, phenoxyethanol, methylparaben, ethylparaben, propylparaben, butylparaben, butylene glycol, a natural tocopherol, glycerol, sodium dihydroxycetyl, isopropyl hydroxycetyl ether, glycol stearate, triisononaoine, octyl cocoate, polyacrylamide, isoparaffin, laureth-7, a carbomer, propylene glycol, glycerol, bisabolol, dimethicone, sodium hydroxide, a perfume, PEG 30-dipolyhydroxystearate, a capric/caprylic triglyceride, cetearyl octanoate, dibutyl adipate, grape seed oil, jojoba oil, magnesium sulfate, EDTA, a cyclomethicone, xanthan gum, citric acid, sodium lauryl sulfate, a mineral wax, a mineral oil, isostearyl isostearate, propylene glycol dipelargonate, propylene glycol isostearate, PEG 8 Beeswax, a hydrogenated palm tree heart oil glyceride, a hydrogenated palm oil glyceride, lanolin oil, sesame oil, cetyl lactate, lanolin alcohol, castor oil, titanium dioxide, a colorant, and a pigment.

12. The composition of claim 1, which is formulated in a form selected from the group consisting of: an aqueous solution, an oily solution, a cream in a pot, a cream in a tube, an aqueous gel, an oily gel, a shower gel, a shampoo, a milk, an emulsion, a microemulsion, a nanoemulsion, an oil-in-water emulsion, a water-in-oil emulsion, a multiple emulsion, a silicone-containing emulsion, a lotion, a liquid soap, a dermatological bar, an ointment, a foam, an anhydrous product, a stick, and a lipstick.

13. The composition of claim 1, wherein said enzyme is grafted by a covalent bond onto a support selected from the group consisting of: a particle which is insoluble in an aqueous phase, and a polymer which is insoluble in an aqueous phase.

14. The composition of claim 1, wherein the enzyme is grafted onto a sphere, the sphere is prepared to react with the enzyme to be grafted by activation with a bifunctional agent.

15. The composition of claim 14, wherein said bifunctional agent is a carbodiimide.

16. The composition of claim 4, wherein said crystallized and cross-linked enzyme is cross-linked by glutaraldehyde.

17. The composition of claim 1, wherein the composition also contains at least one additional active substance, the presence of the enzyme promoting the trans-cutaneous penetration of at least this additional active substance.

18. A method of care selected from the group consisting of: a cosmetic care, and a dermopharmaceutical care, comprising topical application on body areas in need thereof of a composition as defined in claim 1.

19. The method of claim 18, for limiting at least one body reaction selected from the group consisting of: an irritation during topical use of said enzyme, and an allergy during topical use of said enzyme.

20. The method of claim 18, for promoting multiplication of keratinocytes.

21. The method of claim 20, wherein said keratinocytes is selected from the group consisting of: keratinocytes of the skin, and keratinocytes of the hair.

22. The method of claim 18, for performing a whitening effect.

23. The method of claim 20, wherein said enzyme is a protease.

24. The method of 18, to perform an anti-wrinkle effect.

25. The method of claim 18, for promoting the reconstruction of the skin barrier to obtain a barrier effect.

26. The method of claim 25, wherein said enzyme is selected from the group consisting of: a lipase, and an amylase.

27. The method of claim 18, for promoting the elimination of the excess of sebum and the disappearance of the shiny effect of the skin, by topical application on greasy skin areas.

28. The method of claim 27, wherein said enzyme is a lipase.

29. The method of claim 18, for promoting the care of dry skins.

30. The method of claim 29, wherein said enzyme is selected from the group consisting of: a protease, and an amylase.

31. A method of care selected from the group consisting of: a cosmetic care, and a dermopharmaceutical care, comprising topical application on body areas in need thereof of a composition as defined in claim 17, for increasing the trans-cutaneous penetration of at least the active substance contained in this composition.
